(19) United States
(12) Patent Application Publication
(10) Pub. No.: US 2016/0108325 A1
(43) Pub. Date: Apr. 21, 2016

(54) PROCESS FOR HYDROTREATING A COKER KEROSENE STREAM TO PROVIDE A FEED STREAM FOR A PARAFFIN SEPARATION ZONE

(71) Applicant: UOP LLC, Des Plaines, IL (US)

(72) Inventors: Soumendra Mohan Banerjee, New Delhi (IN); Srinivasa Gopalan Varadarajan, Gurgaon (IN)

(21) Appl. No.: 14/518,388

(22) Filed: Oct. 20, 2014

(51) Int. Cl. C10G 67/14; C07C 7/10; C07C 6/06; C07C 5/03; C10G 69/02; C07C 7/00; C07C 7/12

(52) U.S. Cl. CPC C10G 67/14; C07C 7/005; C07C 7/10; C07C 7/12; C07C 5/03; C10G 69/02; C07C 6/06

(57) ABSTRACT

A process for hydrotreating a coker kerosene feed stream to produce a feed stream for a paraffin separation zone. An effluent stream from a hydrotreating zone is passed to an aromatic separation zone to remove aromatics from the effluent stream form the hydrotreating zone and provide an aromatic lean stream. The aromatic lean stream may be passed to the paraffin separation zone to separate normal paraffins from non-normal paraffins. The separated aromatics can be passed to an aromatic processing zone to, for example, to produce xylene or other desirable aromatic hydrocarbons.

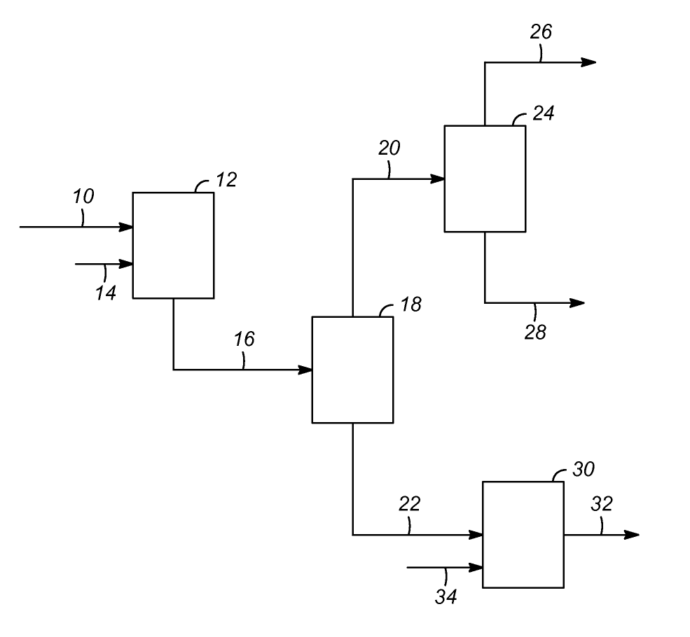

### FIELD OF THE INVENTION

This invention relates generally to hydrotreating a coker kerosene stream, and more particularly to hydrotreating a coker kerosene stream to produce a feed stream for a paraffin separation zone.

### BACKGROUND OF THE INVENTION

Petroleum refiners are keen to upgrade low value coker kerosene to high value feedstock like normal paraffins. Since coker kerosene contains high levels of sulfur and nitrogen, the coker kerosene has to be hydrotreated to reduce the levels of sulfur and nitrogen before the normal paraffins can be separated from the non-normal hydrocarbons in a paraffin separation zone, such as a Molex separation unit from UOP, LLC. (Des Plaines, Il.). Feed stream specifications for such separation zones require severe hydrotreating of the coker kerosene stream to reduce the sulfur to less than 1 wppm and reduce the nitrogen to 0.5 wppm (maximum).

Additionally, one of the feed stream specifications for a paraffin separation zone is that the Bromine Index of the feed stream should be in the range of 50-100. In addition to contaminants like sulfur and nitrogen, coker kerosene also contains olefins, diolefins, and aromatics. A typical aromatics content of coker kerosene is between 20 to 30 wt %. The olefin content is also quite high and is normally designated by the Bromine Number (which is generally 1,000 times the Bromine Index), which is typically between 50 to 55.

Therefore, in order to meet all three feed specifications for sulfur, nitrogen and Bromine Index, coker kerosene is typically subjected to hydrotreating prior to passing to the paraffin separation zone. The hydrotreating conditions can typically involve pressures between 3.4 to 7.6 Mpag (500 to 1100 psig). As will be appreciated, these high pressures consume significant amounts of energy, and include high operating costs.

Therefore, it would be desirable to provide one or more processes which can meet these feed stream specifications for the paraffins separation zone and which allow for hydrotreating at a moderately lower pressure while still recovering the maximum amount of normal paraffins from the coker kerosene stream.

### SUMMARY OF THE INVENTION

One or more processes have been developed for the hydrotreating of a coker kerosene stream to provide a feed for a paraffin separation zone.

In a first aspect of the present invention, the invention may be characterized as a process for the producing a feed for a paraffin separation zone from a coker kerosene feed stream, in which the process comprises: hydrotreating a coker kerosene feed stream to produce a treated coker kerosene stream; separating aromatics from the treated coker kerosene stream to provide an aromatics rich stream and an aromatics lean stream; and, passing the aromatics lean stream to a paraffin separation zone.

In some embodiments, the aromatics are separated from the treated coker kerosene stream with a solvent. It is contemplated that the solvent comprises sulfolane, N-methylpyrrolidone (“NMP”), or an ionic liquid.

In various embodiments, the aromatics lean stream is separated into a normal paraffin rich stream and an iso-paraffin rich stream. It is contemplated that the aromatics lean stream is separated into the normal paraffin rich stream and the iso-paraffin rich stream by a liquid phase adsorption process.

In at least one embodiment, the process may also include recovering an aromatic stream from the aromatic rich stream. The aromatic stream may be rich in xylenes.

In another aspect of the present invention, the invention may be characterized as a process for producing a feed for a paraffin separation zone from a feed stream, in which process includes: reducing a nitrogen and a sulfur content of a coker kerosene feed stream in a hydrotreating zone to produce a treated coker kerosene stream; extracting aromatics from the treated coker kerosene stream in an aromatic extraction zone to provide an aromatics lean stream; and, separating normal paraffins from the aromatics lean stream in a paraffin separation zone to provide a normal paraffin rich stream.

In some embodiments, the process includes decreasing a Bromine Index of the coker kerosene stream in the hydrotreating zone. It is contemplated that the Bromine Index is decreased to between approximately 50 to approximately 100. It is contemplated that decreasing the Bromine Index is performed at a pressure such that the treated coker kerosene stream is not saturated in aromatic compounds.

In at least one embodiment, a solvent used to extract the aromatic compounds is selected from the group consisting of: sulfolane; NMP; and, an ionic liquid.

In some embodiments, the aromatic extraction zone further provides an aromatics rich stream. The process may further include converting toluene from the aromatic rich stream into benzene and xylenes in an aromatics complex to provide a toluene lean aromatic stream.

In still another aspect of the present invention, the invention may be characterized as a process for producing a feed stream to a paraffin separation zone which includes: hydrotreating a coker kerosene feed stream to reduce a nitrogen content and a sulfur content of the coker kerosene feed to provide a treated coker kerosene stream; separating aromatics from the treated coker kerosene stream; and, separating normal paraffins from a portion of the treated coker stream. The aromatics are separated from the treated coker kerosene stream before the normal paraffins are separated from the at least a portion of the treated coker kerosene stream.

In various embodiments, the aromatics are separated from the treated coker kerosene stream by extracting aromatics. It is contemplated that the aromatics are extracted with a solvent selected from the group consisting of: sulfolane; NMP; and, an ionic liquid. It is further contemplated that the process includes passing the solvent and aromatic stream to an aromatics complex and converting toluene into benzene and xylenes in the aromatic complex. It is also contemplated that the normal paraffins are separated from a portion of the treated coker stream by an adsorption process. It is contemplated that the adsorption process is a liquid adsorption.

Additional aspects, embodiments, and details of the invention are set forth in the following detailed description of the invention.

### DETAILED DESCRIPTION OF THE DRAWINGS

The drawing is a simplified process diagrams in which the FIGURE shows one or more processes according to the present invention.

### DETAILED DESCRIPTION OF THE INVENTION

One or more processes have been invented for hydrotreating coker kerosene to provide a feed stream for a paraffin separation zone. As will be explained in more detail below, a coker kerosene stream is first hydrotreated. During the hydrotreating process, sulfur and nitrogen are removed from the coker kerosene. Additionally, diolefins and olefins present in the feed are converted to normal paraffins, increasing the yield of normal paraffins across the hydrotreating step. Typically, this gain is between 4 to 6 wt % for the total normal paraffins present. Thus, hydrotreating provides an incremental increase with respect to the amount of normal paraffins that can be recovered. The effluent stream from the hydrotreating zone is passed to an aromatics separation zone. Since the hydrotreating is carried out at a moderately low pressure, the aromatics in the coker kerosene do not become saturated during the hydrotreating step. This allows the aromatics to be extracted out of the hydrotreated effluent stream to provide an aromatic lean stream which comprises mostly normal paraffins and iso-paraffins. The aromatic lean stream may then be passed to a paraffin separation zone to separate the normal and non-normal paraffins. Since the aromatics have been removed, the paraffin separation zone will process a lower total feed amount and size compared with the same zone processing a feed still containing the aromatics. By reducing the feed, a smaller paraffin separation zone can be used—which can lower capital costs. Additionally, the extracted aromatics can be routed to an aromatics complex to produce desired chemicals, such as xylenes. This provides a refiner with additional value rather than sending the aromatics in the diesel pool. Thus, the present invention is believed to provide for more economical and efficient processes for hydrotreating coker kerosene to provide a feed for a paraffins separation zone.

The various embodiments of the present invention will now be described in detail with reference to the FIGURE, with the understanding the following is merely exemplary of the present invention.

As shown in the FIGURE, a coker kerosene stream 10 is passed to a hydrotreating zone 12. The coker kerosene stream 10 comprises a mixture of C₉ to C₂₀ hydrocarbons. Unlike straight run kerosene, the hydrocarbons in the coker kerosene stream 10 are typically a mixture of both number and type (normal paraffinic, iso-paraffinic, aromatic, mono-olefinic, and diolefinic) of hydrocarbons and because the coker kerosene includes olefins it typically has a Bromine Number between 50 to 55. The coker kerosene stream 10 originates from a coking zone (not shown) in which heavy petroleum residues such as vacuum column residues are thermally cracked to produce lighter hydrocarbons. The thermal cracking conditions including a temperature within the range from about 460 to about 500° C., and a pressure from about 1.0 to 3.0 Kg/cm²g.

In the hydrotreating zone 12, a hydrogen-containing treat gas 14 is used in the presence of suitable catalysts which are primarily active for the removal of heteroatoms, such as sulfur and nitrogen, saturation of olefins and for some hydrogenation of aromatics in from the coker kerosene stream 10.

Suitable hydrotreating catalysts for use in the present invention are any known conventional hydrotreating catalysts and include those which are comprised of at least one Group VIII metal, preferably iron, cobalt and nickel, more preferably cobalt and/or nickel and at least one Group VI metal, preferably molybdenum and tungsten, on a high surface area support material, preferably alumina. Other suitable hydrotreating catalysts include zeolitic catalysts, as well as noble metal catalysts where the noble metal is selected from palladium and platinum. It is within the scope of the present invention that more than one type of hydrotreating catalyst be used in the same reaction vessel. The Group VI metal may be present in an amount ranging from about 2 to about 20 wt %, preferably from about 4 to about 12 wt %. The Group VI metal will typically be present in an amount ranging from about 1 to about 25 wt %, preferably from about 2 to about 25 wt %.

Typical hydrotreating temperatures range from about 204 to 482° C. (400 to 900° F.) with pressures from about 3.6 to 17.3 MPa (500 to 2500 psig), preferably from about 3.6 to 13.9 MPa (500 to 2000 psig). In order to obtain a product stream that has a Bromine Index in the range of 50-100, the operating temperature is preferably in the range of 340 to 370° C. and the pressure can be in the range of 3.6 to 7.6 MPa (500 to 110 psig).

A treated coker kerosene stream 16 is produced by the hydrotreating zone 12. In conventional processes, the treated coker kerosene stream 16 would be passed to a paraffin separation zone; however, according to the present invention, the treated coker kerosene stream 16 is passed first to an aromatic separation zone 18.

In a preferred embodiment, the aromatic separation zone 18 utilizes an aromatic selective solvent, such as sulfolane, N-methylpyrrolidone (“NMP”), or an ionic liquid, to extract aromatic compounds from the treated coker kerosene stream 16 to provide an aromatic lean stream 20 and an aromatic rich stream 22. Aromatic extraction separates, via liquid-liquid extraction or extractive distillation or both, into aromatics and non-aromatics. The development of one commercial process has been reported at the 7th World Petroleum Congress, Volume IV, pages 65-73, 1967, which article is incorporated herein by reference. Conventional processes for the recovery of high purity aromatic hydrocarbons such as benzene, toluene and xylenes (BTX) from various hydrocarbon feeds including catalytic reformate, hydrogenated pyrolysis gasoline, etc., utilize an aromatic selective solvent such as sulfolane, NMP, or an ionic liquid. Typically, in the practice of such processes, a hydrocarbon feed mixture, such as the treated coker kerosene stream 16, is contacted in an extraction zone, such as the aromatic separation zone 18, with an aqueous solvent composition which selectively dissolves the aromatic components from the hydrocarbon feed, thereby forming a raffinate phase comprising one or more non-aromatic hydrocarbons (i.e., the aromatic lean stream 20), and an extract phase comprising solvent having aromatic components dissolved therein (i.e., the aromatic rich stream 22).

The aromatic separation zone 18 may have an operating temperature generally at least about 150° C. (302° F.) and is generally in the range of from about 150° C. (302° F.) to about 275° C. (527° F.).

Returning to the FIGURE, the aromatic lean stream 20 from the aromatic separation zone 18 is passed to the paraffin separation zone 24 that separates normal paraffins from the aromatic lean stream 20 to produce a normal paraffin stream 26. The paraffin separation zone 26 also produces a non-paraffin rich stream 28. The non-paraffin rich stream 28 can include naphthenes, iso-paraffins, and other non-normal hydrocarbons.

The paraffin separation zone 26 can separate normal paraffins from the aromatic lean stream 20 by any suitable process, such as, for example, by a liquid-phase adsorptive separation process. Several known processes that accomplish such a separation are known. One process, the Molex process, is an established, commercially proven method for the liquid-phase adsorption separation of normal paraffins from isoparaffins, cycloparaffins, and aromatics using the Sorbex separation technology. See Chapters 10.3 and 10.7 in the book entitled Handbook of Petroleum Refining Process, Second Edition, edited by Robert A. Meyers, published by McGraw-Hill, N.Y., 1997. This separation can be performed in a batch or continuous mode including the use of two or more adsorbent beds in cyclic operation. In this mode one or more beds are used for the separation while another bed is being regenerated. Significant operational and economic advantages accrue to performing the separation on a continuous basis which produces a product of uniform composition. The preferred method of achieving continuous operation and uniform products is by the use of simulated moving bed technology.

The normal paraffin stream 26 from the paraffin separation zone 24 can be passed to one or more fractionation columns (not shown) to separate out one or more fractions of the normal paraffins. As previously mentioned, since the feed stream (i.e., the aromatic lean stream 20) to the paraffin separation zone 24 contains fewer aromatics, the paraffin separation zone 24 will process a lower total feed amount and size, allowing for a smaller separation zone compared to a typical zone separation zone associated with conventional processes.

The non-normal paraffin rich stream 28 from the paraffin separation zone 24 may be routed to a transportation fuel pool (i.e., jet fuel or diesel) based on demand.

In addition to the capital savings associated with having a smaller paraffin separation zone 24, the processes according to the present invention may allow for a refiner to convert the aromatics from the aromatic rich steam 22 into more valuable chemical compounds. Accordingly, the aromatic rich steam 22 may be passed to an aromatics processing zone 30 to convert a portion of the aromatic compounds into a desired aromatic product stream 32. For example, the desired aromatic product stream 32 may comprise benzene, toluene, xylenes or a combination thereof, and may be passed to a downstream aromatic complex for the recovery of the different aromatics.

In an exemplary aromatics processing zone 30, as is known, the feed stream (i.e., the aromatic rich steam 22) to the aromatics processing zone 30 may be first heated by indirect heat exchange against the reaction effluent stream and then is heated to reaction temperature by exchange with a warmer stream, steam or a furnace. A hydrogen stream 34 may be introduced to the aromatics processing zone 30. The hydrogen stream 30 may comprise other compounds, e.g. C₁ to C₄ hydrocarbons, in addition to hydrogen. The hydrogen and hydrocarbons may be recycled in the process as described below. If present, free hydrogen is associated with the feedstock and recycled hydrocarbons, if any, in an amount from about 0.1 moles per mole of aromatics up to 10 moles per mole of aromatics. This ratio of hydrogen to aromatics is also referred to as hydrogen to hydrocarbon ratio.

The aromatic processing zone 30 comprises one more reactors containing a catalyst to produce a reaction effluent stream comprising unconverted and desired product hydrocarbons, for example xylenes and benzene. For example, the aromatic processing zone 30 can perform known processes for toluene disproportionation and transalkylation with C₉ or C₁₀₊ aromatic hydrocarbons to produce benzene and xylenes. Additionally and alternatively, the aromatic processing zone 30 can isomerize xylene to an equilibrium mixture and convert ethyl benzene to benzene or xylenes.

As mentioned above, this reaction effluent stream is normally cooled by indirect heat exchange against the aromatic feed stream entering the aromatic processing zone 30 and may be further cooled through the use of air or cooling water. The reaction effluent stream may be separated, for example in a vapor-liquid separator, to produce a vapor phase hydrogen stream and a liquid phase reaction effluent stream. The vapor phase hydrogen stream includes hydrogen and light hydrocarbons which may be recycled and combined with the feed as described above. The liquid phase reaction effluent stream may be passed into a stripping column in which substantially all C₅ and lighter hydrocarbons present are concentrated into an overhead stream and removed from the process. The stripping column also produces a net stripper bottoms stream, which is the aromatic processing zone effluent stream.

The aromatic processing zone effluent stream may be further separated in a distillation zone comprising at least one distillation column to produce one or more product streams, such as the desired aromatic product stream 32. Various flow schemes and combinations of distillation columns to separate the aromatic processing zone effluent stream via fractional distillation are well known in the art. See, e.g., U.S. Pat. No. 7,605,295. Additionally, another such process for separating the components of the aromatic processing zone effluent stream is a liquid adsorption process, and more particularly, a simulated moving bed liquid adsorption process, such as the Parex process. Briefly, in such a process a feed stream, such as the aromatic processing zone effluent stream, is passed into contact with an adsorbent. An extract component will selectively be adsorbed by the adsorbent, while a raffinate component will be less selectively adsorbed. A raffinate stream can be removed, and desorbent can be used to remove, or desorb, the extract components from the adsorbent in an extract stream. Again, such separation process are known. See, e.g., U.S. Pat. No. 3,997,620 and U.S. Pat. No. 4,326,092. The type of process used to separate the components of the aromatic processing zone effluent stream are not necessarily important to the practicing of the present invention.

In conventional processes in which the aromatics were contained in the non-normal paraffin stream 28 from a paraffin separation zone 24, the aromatics would have been passed to a transportation fuel blending pool. However, in the various processes according to the present invention, the aromatics can be converted into more desired products, which may be more economical for the refiner.

Thus, the present invention provides various efficient and economical processes for producing a feed stream for a paraffin separation zone from coker kerosene.

It should be appreciated and understood by those of ordinary skill in the art that various other components such as valves, pumps, filters, coolers, etc. were not shown in the drawings as it is believed that the specifics of same are well within the knowledge of those of ordinary skill in the art and a description of same is not necessary for practicing or understating the embodiments of the present invention.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A process for the producing a feed for a paraffin separation zone from a coker kerosene feed stream, the process comprising:
hydrotreating a coker kerosene feed stream to produce a treated coker kerosene stream;
separating aromatics from the treated coker kerosene stream to provide an aromatics rich stream and an aromatics lean stream; and,
passing the aromatics lean stream to a paraffin separation zone.

2. The process of claim 1 wherein the aromatics are separated from the treated coker kerosene stream with a solvent.

3. The process of claim 2 wherein the solvent comprises sulfolane, NMP, or an ionic liquid.

4. The process of claim 1 further comprising:
separating the aromatics lean stream into a normal paraffin rich stream and an iso-paraffin rich stream.

5. The process of claim 4 wherein the aromatics lean stream is separated into the normal paraffin rich stream and the iso-paraffin rich stream by a liquid phase adsorption process.

6. The process of claim 1 further comprising:
recovering an aromatic stream from the aromatic rich stream.

7. The process of claim 6 wherein the aromatic stream is rich in xylenes.

8. A process for producing a feed for a paraffin separation zone from a feed stream, the process comprising:
reducing a nitrogen and a sulfur content of a coker kerosene feed stream in a hydrotreating zone to produce a treated coker kerosene stream;
extracting aromatics from the treated coker kerosene stream in an aromatic extraction zone to provide an aromatics lean stream; and,
separating normal paraffins from the aromatics lean stream in a paraffin separation zone to provide a normal paraffin rich stream.

9. The process of claim 8 further comprising:
decreasing a Bromine Index of the coker kerosene stream in the hydrotreating zone.

10. The process of claim 9 wherein the Bromine Index is decreased to between approximately 50 to approximately 100.

11. The process of claim 9 wherein the step of decreasing the Bromine Index is performed at a pressure such that the treated coker kerosene stream is not saturated in aromatic compounds.

12. The process of claim 8 wherein a solvent used to extract the aromatic compounds is selected from the group consisting of: sulfolane; NMP; and, an ionic liquid.

13. The process of claim 8 wherein the aromatic extraction zone further provides an aromatics rich stream.

14. The process of claim 13 further comprising:
converting toluene from the aromatic rich stream into benzene and xylenes in an aromatics processing zone to provide a toluene lean aromatic stream.

15. A process for reducing a feed stream to a paraffin separation zone, the process comprising:
hydrotreating a coker kerosene feed stream to reduce a nitrogen content and a sulfur content of the coker kerosene feed to provide a treated coker kerosene stream;
separating aromatics from the treated coker kerosene stream; and,
separating normal paraffins from a portion of the treated coker stream,
wherein the aromatics are separated from the treated coker kerosene stream before the normal paraffins are separated from the at least a portion of the treated coker kerosene stream.

16. The process of claim 15 further comprising:
separating aromatics from the treated coker kerosene stream by extracting aromatics.

17. The process of claim 16 wherein the aromatics are extracted with a solvent selected from the group consisting of: sulfolane; NMP; and, an ionic liquid.

18. The process of claim 17 further comprising:
passing the solvent and aromatic stream to an aromatics processing zone;
converting toluene into benzene and xylenes in the aromatic processing zone.

19. The process of claim 18 further comprising:
separating normal paraffins from a portion of the treated coker stream by an adsorption process.

20. The process of claim 19 wherein the adsorption process is a liquid adsorption.
